(19) United States
(12) Patent Application Publication
(10) Pub. No.: US 2012/0289631 A1
(43) Pub. Date: Nov. 15, 2012

(54) AROMATIC DIAMINE AND METHOD FOR MANUFACTURING THE SAME, AND ARAMID FIBER AND METHOD FOR MANUFACTURING THE SAME

(76) Inventors: Hyojin Lee, Gyeongsangbuk-do (KR); In Sik Han, Daegu (KR)

(21) Appl. No.: 13/519,760

(22) PCT Filed: Dec. 29, 2010
(86) PCT No.: PCT/KR2010/009512

§ 371 Date: Jun. 28, 2012

(30) Foreign Application Priority Data
Dec. 29, 2009 (KR) 10-2009-0133031
Dec. 31, 2009 (KR) 10-2009-0135807

(51) Int. Cl. C07C 211/51; C08K 5/50; B29C 47/00; C08K 5/3435; C08L 77/00; C07C 209/84; C08K 5/134

(52) U.S. Cl. 524/99; 564/305; 564/437; 524/154; 524/291; 264/8

(57) ABSTRACT

Disclosed is aromatic diamine with high purity obtained by efficiently removing impurities and preventing oxidation, and a method for manufacturing the same; and aramid fiber with improved discoloration resistance realized by preventing deterioration in quality even for a long-period exposure to the external environments of sunlight, atmosphere, and moisture, and a method for manufacturing the same.

### TECHNICAL FIELD

The present invention relates to aromatic diamine and a method for manufacturing the same, and aramid fiber and a method for manufacturing the same, and more particularly, to aromatic diamine with improved property and discoloration resistance, and a method for manufacturing the same, and aramid fiber using the aromatic diamine with improved mechanical property and discoloration resistance, and a method for manufacturing the same.

### BACKGROUND ART

Generally, fully aromatic polyamide fiber commonly known as aramid fiber may be classified into p-aramid fiber and m-aramid fiber, wherein the p-aramid fiber is made in such a structure that benzene rings are linearly connected through an amide group (CONH). At this time, the p-aramid yarn of 5 mm diameter has such a strength as to lift up and maintain a two-ton car. Thus, the p-aramid fiber is used in various fields for advanced technology of aerospace industry as well as the industry for developing a bullet-resistant material. Also, since the aramid fiber is carbonized at a temperature above 500° C., the aramid fiber has attracted great attentions in the field requiring high heat resistance.

A process for manufacturing the aramid fiber includes steps of preparing fully aromatic polyamide polymer by polymerizing aromatic diamine and aromatic diacid halide in a polymerization solvent containing N-methyl-2-pyrrolidone (NMP); preparing a spinning dope by dissolving the aromatic polyamide polymer in a concentrated sulfuric acid solution; preparing a filament by extruding the spinning dope through the use of spinneret, and making the extruded spinning dope pass through non-coagulation fluid and coagulation bath; and washing, drying and heat-treating the prepared filament.

However, since the prior art aromatic diamine has disadvantages of low purity degree and high reactivity (which might be easily deteriorated by the exposure to oxygen), the aramid polymer prepared with the prior art aromatic diamine has a low and non-uniform molecular weight. Thus, the aramid fiber prepared using the prior art aromatic diamine has problems of discoloration and deteriorated property.

Also, if the prior art aramid fiber is exposed to the external environments of sunlight, atmosphere, and moisture for a long time, the property of the prior art aramid fiber is deteriorated by discoloration.

### DISCLOSURE OF INVENTION

### Technical Problem

Therefore, the present invention has been made in view of the above problems, and it is an object of the present invention to provide aromatic diamine and a method for manufacturing the same, and aramid fiber and a method for manufacturing the same, which is capable of preventing one or more problems of the prior art.

The object of the present invention is to provide aromatic diamine which has high purity degree obtained by removing impurities and preventing oxidation, and a method for manufacturing the same.

Another object of the present invention is to provide aramid fiber which has improved discoloration resistance by preventing deterioration in quality even for a long-period exposure to the external environments of sunlight, atmosphere, and moisture, and a method for manufacturing the same.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

### Solution to Problem

To achieve these objects and other advantages and in accordance with the purpose of the invention, as embodied and broadly described herein, there is provided aromatic diamine containing 15 ppm or less of impurities.

In another aspect of the present invention, there is provided aramid fiber comprising:

aromatic polyamide polymer by polymerizing high-purity aromatic diamine with aromatic diacid halide; and stabilizer added to the aromatic polyamide polymer, wherein a color retention (ΔL) is −18.0˜−12.0.

In another aspect of the present invention, there is provided a method for manufacturing aromatic diamine comprising: adding a refining supplement to aromatic diamine; and refining the aromatic diamine with the refining supplement.

In another aspect of the present invention, there is provided a method for manufacturing aramid fiber comprising: preparing high-purity aromatic diamine; preparing aromatic polyamide polymer by polymerizing the aromatic diamine with aromatic diacid halide; preparing spinning dope by dissolving the aromatic polyamide polymer in a solvent; and preparing aramid filament by spinning the spinning dope.

### Advantageous Effects of Invention

The aromatic diamine according to the present invention and the method for manufacturing the same, and the aramid fiber according to the present invention and the method for manufacturing the same have the following advantages.

First, it is possible to obtain the aromatic diamine with high purity degree by removing the impurities and preventing the oxidation. The aromatic diamine with high purity degree may be applied in various fields requiring the high property and good color.

Also, even though the aramid fiber according to the present invention is exposed to the external environments of sunlight, atmosphere, and moisture for a long time, it is possible to obtain the aramid fiber having the improved discoloration resistance and property. The aramid fiber having the good properties may be used in the various fields.

### BEST MODE FOR CARRYING OUT THE INVENTION

Reference will now be made in detail to the preferred embodiments of the present invention.

First, the aromatic diamine according to the present invention and the method for manufacturing the same will be described as follows.

The aromatic diamine may be used in various fields, especially, in the field for preparing aramid polymer. The aramid polymer is prepared by polymerizing aromatic diamine with aromatic diacid halide. For preparing the aramid polymer with high and uniform molecular weight, it is necessary to obtain high purity degree in the aromatic diamine and aromatic diacid halide.

The aromatic diamine, which is one of ingredients in the aramid polymer, contains a large amount of impurities, generally. The impurities may contain chloroaniline capable of functioning as a polymerization terminator. Thus, if the aromatic diamine contains the large amount of impurities such as chloroaniline, the aramid polymer prepared with the aromatic dimaine containing the large amount of impurities has the low and non-uniform molecular weight.

Accordingly, the purity of aromatic diamine largely affects the property of final product. That is, the aramid polymer prepared with the low-purity aromatic diamine has the low and non-uniform molecular weight, the final product using the aramid polymer prepared with the low-purity aromatic diamine has the problem of deteriorated property. For example, if the aramid fiber is manufactured with the aramid polymer prepared with the low-purity aromatic diamine, bulletproof vest using the aramid fiber prepared with the low-purity aromatic diamine cannot realize high bulletproof performance, and thus cannot ensure security of the human body.

In order to prepare the aromatic diamine with high purity, a method for efficiently refining the aromatic diamine will be described as follows.

The aromatic diamine may be smoothly refined in a heat distillation method.

The aromatic diamine may contain p-phenylenediamine or m-phenylenediamine. Herein, the boiling point of p-phenylenediamine is 267° C., and the boiling point of m-phenylenediamine is 284˜287° C.

The impurities contained in the aromatic diamine may contain aniline or p-chloroaniline. Herein, the boiling point of aniline is 184.4° C., and the boiling point of p-chloroaniline is 232° C.

There is a large difference in boiling point between the aromatic diamine and the impurities such as aniline, whereby it is possible to separate the impurities from the aromatic diamine by the aforementioned heat distillation method with easiness.

For separation of the impurities and aromatic diamine having the different boiling points from each other, the heat distillation method is carried out by heating the aromatic diamine in a distillation column. That is, the aromatic diamine having the high boiling point is positioned at a lower portion of the distillation column, and the impurities having the low boiling point are positioned at an upper portion of the distillation column. Thus, separate pipes are respectively connected with the lower and upper portions of the distillation column, to thereby separate the impurities from the aromatic diamine.

However, if there is a small difference in boiling point between the aromatic diamine and the impurities, it is difficult to obtain the aromatic diamine with the required purity degree by one refining process. In order to obtain the aromatic diamine with the required purity degree, the aromatic diamine may be refined two or three times. However, the repetitive refining process may cause the largely-increased cost and make it difficult to obtain the aromatic diamine with high purity.

For realizing the simplified process and obtaining the aromatic diamine with high purity, a refining supplement may be added to the aromatic diamine before the refining process.

The refining supplement may be an organic solvent. The organic solvent enables to easily separate the impurities from the aromatic diamine.

Preferably, the boiling point of organic solvent is lower than the boiling point of aromatic diamine. Owing to the organic solvent whose boiling point is lower than the boiling point of aromatic diamine and is similar to the boiling point of impurities, the impurities are easily separated from the aromatic diamine.

The organic solvent may be water; methanol; ethanol; benzene; toluene; N-methyl-2-pyrrolidone (NMP); N,N′-dimethylacetamide (DMAc); N,N-dimethylformamide (DMF); or dimethyl sulfoxide (DMSO).

In comparison to the weight of impurities, the organic solvent may be added in a range from 1 time to 100,000 times. If the weight of the added organic solvent is less than 1 time, the impurities may not be separated from the aromatic diamine. Meanwhile, if the weight of the added organic solvent is more than 100,000 times, the manufacturing cost is increased without improvement of the refining performance.

In the present invention, since the organic solvent functioning as the refining supplement is added into the aromatic diamine, the required aromatic diamine with high purity can be obtained by one refining process, to thereby reduce the cost and time consumed in the refining process.

Herein, the concentration of chloroaniline impurity contained in the aromatic diamine prepared by the aforementioned process may be not more than 5 ppm. The chloroaniline contains p-chloroaniline.

If the aromatic diamine is refined by the heat distillation method, the aromatic diamine is exposed to the atmosphere containing oxygen at a high temperature. If the aromatic diamine is exposed to the atmosphere at a high temperature, the aromatic diamine easily reacts with the oxygen due to the high reactivity, and is oxidized, whereby it might be changed to nitro compounds. As the oxidized aromatic diamine functions as the impurities, the aramid polymer prepared with the aromatic diamine containing the oxidized aromatic diamine is heavily colored, to thereby cause the deteriorated color property. Thus, if the aramid polymer with the deteriorated color property is used for manufacturing the products, the customer's reliability may be deteriorated due to discoloration of the products.

In order to prevent the aromatic diamine from being oxidized, an antioxidant corresponding to the refining supplement is added into the aromatic diamine before refining the aromatic diamine. The antioxidant prevents the aromatic diamine from being in contact with the oxygen for the refining process. That is, the antioxidant more easily reacts with the oxygen since reactivity of the antioxidant is better than reactivity of the aromatic diamine. Thus, it is possible to prevent the reaction between the aromatic diamine and the oxygen, to thereby prevent the aromatic diamine from being oxidized.

The antioxidant may contain hydrazine. The antioxidant containing the hydrazine removes the oxygen by the following reaction formula 1.

N₂H₄+O₂→N₂+2H₂O [Reaction Formula 1]

Also, the antioxidant may contain hydrazine compounds expressed by the following chemistry figure 1.

ChemistryFigure 1

R₁R₂N—NR₃R₄ [Chem.1]

At this time, ‘R1’, ‘R2’, ‘R3’, and ‘R4’ may be aliphatic group, aromatic group, halogen atom, hydroxyl group, nitro group, nitroso grouop, cyano group, amino group, imino group, azo group, carbonyl group, carboxyl group, acyl group, alkoxycarbonyl group, aryloxycarbonyl group, carbamoyl group, alkoxyl group, aryloxy group, haloalkyl group, mercapto group, alkylthio group, arylthio group, sulfo group, sulphinyl group, sulfonyl group, or heterocyclic group, respectively. Also, two substituents selected from ‘R1’, ‘R2’, ‘R3’, and ‘R4’ may be cyclic compounds combined discretionally.

The aromatic diamine refined after addition of the antioxidant may contain 10 ppm or less of the oxidized aromatic diamine.

The aromatic diamine refined after addition of the antioxidant may contain p-phenylenediamine; m-phenylenediamine; 4,4′-diaminobiphenyl; 1,3-diaminobiphenyl; 2,6-naphthalenediamine; 1,5-naphthalenediamine; or 4,4′-diaminobenzanilide.

The aromatic diamine refined after addition of the refining supplement may contain 15 ppm or less of the impurities.

Aramid polymer and a method for manufacturing the same will be described as follows.

First, a mixture solution is prepared by dissolving the aforementioned aromatic diamine refined to have the high-purity degree in a polymerization solvent.

The polymerization solvent is prepared by adding inorganic salt into an organic solvent. The organic solvent may be an amide-based organic solvent, a urea-based organic solvent, or their mixture, for example, N-methyl-2-pyrrolidone (NMP); N, N′-dimethylacetamide (DMAc); hexamethylphosphoramide (HMPA); N,N,N′,N′-tetramethyl urea (TMU); N,N-dimethylformamide (DMF); or their mixtures.

The aromatic diamine is apt to discolor by the reaction with the atmosphere, and the discolored aromatic diamine functions as the impurities. The aramid polymer manufactured by the aromatic diamine containing the discolored aromatic diamine may have the lowed molecular weight and deteriorated color property.

In order to prevent the discoloration of the aromatic diamine, a stabilizer is added into the aromatic diamine according to the present invention. The stabilizer may contain organic phosphorous-based stabilizer, phenol-based stabilizer, and hindered amine-based stabilizer. Especially, the stabilizer using phosphine-based compounds may stabilize the aromatic diamine.

The phosphine-based stabilizer may contain ethyl phosphine; n-butyl phosphine; phenyl phosphine; diethyl phosphine; di(n-butyl) phosphine; diphenylphosphine; triethylphosphine; tri(n-butyl) phosphine; tri(cyclohexyl)phosphine; or triphenylphosphine.

Even though the aromatic diamine is exposed to the external environments of atmosphere and moisture, the stabilizer protects the aromatic diamine from the external environments, to thereby prevent the discoloration of the aromatic diamine.

Then, a predetermined amount of aromatic diacid halide is added to the mixture solution while stirring the mixture solution, thereby resulting in preliminary polymerization.

According to one embodiment of the present invention, the preliminary polymerization is carried out while being maintained at 0˜45° C. temperature for 3˜15 minutes. For the preliminary polymerization, it is preferable to add only 20˜40% of the aromatic diacid halide among the entire weight of aromatic diacid halide to be required for manufacturing the fully aromatic polyamide polymer.

After completing the preliminary polymerization, the temperature is lowered to 1˜10° C., the remaining aromatic diacid halide is added to the preliminary polymer, to thereby prepare the final polymer. The detailed example of the aromatic polyamide polymer finally obtained by the polymerization process may be poly(paraphenylene terephtalamide: PPD-T); poly(4,4′-benzanilide terephtalamide); poly(paraphenylene-4,4′-biphenylene-dicarboxyl acid amide); or poly(paraphenylene-2,6-naphthalenedicarboxyl acid amide).

Then, an alkali compound is used to neutralize the acid produced for the polymerization reaction. The alkali compound may be selected from groups of carbonate of alkali metal or alkali earth metal, hydride of alkali earth metal, hydroxide of alkali earth metal, or oxide of alkali earth metal, for example, NaOH, Li₂CO₃, CaCO₃, LiH, CaH₂, LiOH, Ca(OH)₂, Li₂O, or CaO.

After that, the polymerization solvent is extracted from the neutralized aromatic polyamide polymer. Since the polymerization solvent used for the polymerization process is contained in the aromatic polyamide polymer obtained by the polymerization, the polymerization solvent has to be extracted from the aromatic polyamide polymer, and the extracted polymerization solvent has be to re-used for the polymerization process. The extracting process using water is the most economical and efficient.

After the extracting process, a dehydrating process is performed so as to remove the remaining water. Then, the aromatic polyamide polymer is completed through a drying process.

Next, aramid fiber and a method for manufacturing the same will be described as follows.

First, a spinning dope is prepared by dissolving the aromatic polyamide polymer prepared by the aforementioned method in a solvent of concentrated sulfuric acid having a concentration of 97 to 100%.

At this time, the stabilizer may be added to the spinning dope instead of the aforementioned aromatic diamine. If it is possible to prevent the refined aromatic diamine with high purity from being in contact with the oxygen for the polymerization process, addition of the stabilizer to the spinning dope is more economical, and is more efficient in preventing the discoloration of the aramid fiber. That is, if the stabilizer is added to the spinning dope, it is possible to minimize the loss of stabilizer, and to uniformly distribute the stabilizer in the polymer, to thereby maximize the performance of preventing the discoloration in aramid fiber.

After spinning the spinning dope through the use of spinneret, the spinning dope passing through an air gap is coagulated in a coagulation bath filled with a coagulation solution, to thereby prepare filament.

Then, the remaining sulfuric acid is removed from the prepared filament. Most of the sulfuric acid used for manufacturing the spinning dope is removed when the spinning dope passes through the coagulation bath. However, since it is difficult to completely remove the sulfuric acid, the sulfuric acid might remain in the prepared filament. Also, if the sulfuric acid is added to the coagulation solution in the coagulation bath so as to uniformly extract the sulfuric acid from the spinning dope, there is a high possibility of the sulfuric acid remaining in the prepared filament. Even though a small amount of sulfuric acid remains in the prepared filament, it has a bad influence on the property of aramid fiber. Thus, it is very important to completely remove the sulfuric acid from the filament. The sulfuric acid remaining in the filament may be removed by a washing process using water, or mixture of water and alkali solution.

The washed aramid filament is dried. In this case, the moisture content of the aramid filament may be determined by adjusting a contact time between a drying roll and the filament, or adjusting a temperature of the drying roll.

Then, the completely-dried filament is wound on a winder, to thereby complete the aramid fiber.

The aramid fiber contains plural amine or acid end groups. Especially, since the amine end groups have high reactivity, the amine end groups are apt to be changed by the long-time exposure to the external environments of sunlight, atmosphere, moisture, and etc., whereby the aramid fiber is considerably deteriorated in color property.

Accordingly, the aramid fiber of the present invention contains the stabilizer.

The process of adding the stabilizer to the aramid fiber may be carried out during the polymerization process or spinning process, or may be carried out before the winding process of the prepared aramid filament.

If the process of adding the stabilizer to the aramid fiber is carried out before the winding process, it uses a filament-manufacturing apparatus, whereby it has advantages of good productivity, lowered manufacturing cost, and uniform distribution of the stabilizer in the aramid filament.

A method of adding the stabilizer to the aramid fiber may be carried out in various ways, for example, dipping method, roller method, spray method, and etc.

The aramid fiber prepared by the aforementioned process may contain 20 to 10,000 ppm of the stabilizer. If the content of stabilizer is less than 20 ppm, it is difficult to prevent the discoloration of the aramid fiber. Meanwhile, if the content of stabilizer is more than 10,000 ppm, the crystalinity of the aramid fiber is deteriorated due to the large amount of stabilizer without the large improvement in performance of preventing the discoloration of the aramid fiber.

The aramid fiber with the stabilizer, which is prepared through the use of refined aromatic diamine with high purity, realizes good discoloration resistance so that the aramid fiber maintains −18.0˜−12.0 color retention.

### EMBODIMENT 1

The organic solvent of toluene, functioning as the refining supplement, is added to p-phenylenediamine with 98? purity, wherein the content of organic solvent is 10 times in comparison to the content of impurities of the p-phenylenediamine. Then, the mixture of organic solvent and p-phenylenediamine is heat-distilled in the distillation column, to thereby obtain refined p-phenylenediamine.

### EMBODIMENTS 2 TO 4

P-phenylenediamine is obtained in the same method as the aforementioned first embodiment except that the content of added organic solvent, that is, the content of added toluene is 1 time, 100 times, and 1,000 times in comparison to the content of impurities.

### COMPARATIVE EXAMPLE 1

P-phenylenediamine is obtained in the same method as the aforementioned first embodiment except that the organic solvent, that is, toluene is not added.

The respective purities of p-phenylenediamine obtained by the aforementioned embodiments 1 to 4 and comparative example 1 are measured by the following method, and measurement results are shown in the following Table 1.

### Measuring Purity (%) of P-Phenylenediamine

The purity of p-phenylenediamine (PPD) is measured by a quantitative analysis under the following conditions and procedure through the use of gas chromatography (GC).

The measuring conditions of gas chromatography (GC) are shown as follows.

1) Measuring apparatus: Agilent 6890

2) Integration: GC Chemstation

3) Carrier gas: He

4) Flow: 1.5 ml/minute

5) Split ratio: 10:1 (15 ml/minute)

6) Injection concentration: 10 wt % SMPL in 99.9% of methanol

7) Column: Rtx-1701, 30 m×0.32 mm×1.0 μm

8) Temperature profile: injector 280° C., detector 280° C., 40 minutes×140° C., 30° C./minute to 240° C., 20 minutes×240° C.

A procedure of measuring the gas chromatography (GC) is explained as follows.

1) 10 wt % sample is prepared by completely melting the prepared p-phenylenediamine in a reagent of methanol having 99.9% purity.

2) The sample is injected through the use of syringe, and the quantitative analysis is made under the aforementioned conditions.

TABLE 1

| | Toluene addition (times) | Purity (%) |
|---|---|---|
| Embodiment 1 | 10 | 99.9995 |
| Embodiment 2 | 1 | 99.9995 |
| Embodiment 3 | 100 | 99.9996 |
| Embodiment 4 | 1000 | 99.9996 |
| Comparative | 0 | 99.9989 |
| example 1 | | |

### EMBODIMENT 5

The mixture is prepared by adding the organic solvent of toluene to the aromatic diamine of p-phenylenediamine, and subsequently adding the antioxidant of hydrazine thereto, wherein the concentration of toluene is 50 weight % of p-phenylenediamine, and the concentration of hydrazine is 5 weight % of p-phenylenediamine. Then, the mixture is heat-distilled in the distillation column, to thereby obtain refined p-phenylenediamine.

### EMBODIMENTS 6 TO 10

Refined p-phenylenediamine is obtained in the same method as the aforementioned fifth embodiment except that the concentration of hydrazine corresponding to the antioxidant is changed to 1, 20, 50, 70, and 90 weight % in comparison to the concentration of p-phenylenediamine.

The respective contents of oxidized aromatic diamine and chloroaniline of p-phenylenediamine obtained by the aforementioned embodiments 5 to 10 and comparative example 1 are measured by the aforementioned method of measuring the purity of p-phenylenediamine, and measurement results are shown in the following Table 2.

TABLE 2

| | | | Content of |
|---|---|---|---|
| | concentration of | Content of | Oxidized |
| | hydrazine | Chloroaniline | aromatic diamine |
| | (weight %) | (ppm) | (ppm) |
| Embodiment 5 | 5 | 4 | 10 |
| Embodiment 6 | 1 | 4 | 10 |
| Embodiment 7 | 20 | 4 | 9 |
| Embodiment 8 | 50 | 5 | 9 |
| Embodiment 9 | 70 | 5 | 9 |
| Embodiment 10 | 90 | 5 | 9 |
| Comparative | 0 | 37 | 618 |
| example 1 | | | |

### Preparing Aramid Fiber

### EMBODIMENT 11

The mixture solution is prepared by adding 5.00 g of p-phenylenediamine containing 1,000 ppm of tri-phenylphosphine stabilizer, which is refined by the method of the aforementioned embodiment 5, to 100 ml of polymerization solvent obtained by adding 7 weight % of CaCl₂ to N-methyl-2-pyrrolidone (NMP). Then, 3.18 g of terephthaloyl dichloride is added to the mixture solution, and is then reacted for 6 minutes under 500 rpm rotary power, to thereby prepare the preliminary polymer. Thereafter, 5.61 g of terephthaloyl dichloride is added to the preliminary polymer, and is then reacted for 15 minutes, to thereby prepare poly(paraphenylene terephtalamide). Then, the spinning dope is prepared by dissolving the prepared poly(paraphenylene terephtalamide in 98% concentrated sulfuric acid.

After the spinning dope extrudes through the spinneret, the spinning dope is coagulated in the coagulation bath by passing through the air gap, to thereby obtain aramid filament. Then, the aramid filament is washed by the alkali solution.

The washed aramid filament is dried at 150%° C. for 3 seconds, and then the dried aramid filament is wound on a bobbin, to thereby complete aramid fiber.

### EMBODIMENT 12

Aramid fiber is prepared in the same method as the aforementioned embodiment 11 except that phenol-based Irganox1010 (CIBA Chem.) stabilizer is used instead of the tri-phenylphosphine stabilizer.

### EMBODIMENT 13

Aramid fiber is prepared in the same method as the aforementioned embodiment 11 except that hindered amine-based Tinuvin-292 (CIBA Chem.) stabilizer is used instead of the tri-phenylphosphine stabilizer.

### EMBODIMENTS 14 TO 19

Aramid fiber is prepared in the same method as the aforementioned embodiment 11 except that the content of stabilizer added to p-phenylenediamine is changed to 10 ppm, 500 ppm, 3000 ppm, 6000 ppm, 9000 ppm, and 12000 ppm.

### EMBODIMENT 20

Aramid fiber is prepared in the same method as the aforementioned embodiment 11 except that dried aramid filament is embedded in a stabilizing solution, and is wound therein. At this time, the concentration of stabilizing solution is 10 weight %.

### COMPARATIVE EXAMPLE 2

Aramid fiber is prepared in the same method as the aforementioned embodiment 11 except that the stabilizer is not added to the aromatic diamine.

Color retention, strength retention, and modulus retention of the aramid fibers obtained by the aforementioned embodiments 11 to 20 and comparative example 2 are measured by the following method, and measurement results are shown in the following table 3.

### Measuring Color Retention (ΔL)

The color of aramid fiber is measured by COLOR-7X (KURABO Industries Ltd.), and the measured color value is defined as ‘L’. In this case, ‘color change (ΔL)’ is measured after the aramid fiber having an initial color ‘L1’ is left 24 hours under the conditions of black panel temperature (65±3° C.), exposure light source (Xenon-Arc), irradiance (0.35 W/m²×340 nm) and exposure cycle (102 min of light only/18 min light and water spray). At this time, a sample is prepared by winding the aramid fiber on a paper tube having 107 mm diameter at a uniform curvature, wherein a winding width of aramid fiber on the paper tube is 190 mm, and a total weight of aramid fiber is 5 kg. Then, the color value is measured at each of 10 points of the prepared sample, and the average value of 8 color values is calculated without the maximum and minimum color values, to thereby obtain the color of aramid fiber.

### Measuring Strength Retention (%) and Modulus Retention (%)

The strength retention and modulus retention are obtained by measuring the strength (g/d) of aramid fiber and the modulus (g/d) of aramid fiber, respectively before and after the conditions of Q-UV apparatus (CLEVELAND, 26200 First), 45° C. temperature, and 1008 hours.

In more detail, a force (g) is measured through the use of Instron tester (Instron Engineering Corp, Canton, Mass.) when the sample having 25 cm length is broken. Then, the measured force is divided by a denier of the sample, thereby measuring the strength and modulus. In this case, an extension speed is 300 mm/minute, and a preliminary tension is ‘fineness× 1/30 g’. After the strength and modulus are measured with 5 samples of aramid fiber, the average value of 5 samples is calculated.

Strength retention (%)=(strength measured after the aramid after is left under the aforementioned conditions/strength measured before the aramid is left under the aforementioned conditions)×100

Modulus retention (%)=(modulus measured after the aramid after is left under the aforementioned conditions/modulus measured before the aramid is left under the aforementioned conditions)×100

TABLE 3

| | | Strength | | Modulus | |
|---|---|---|---|---|---|
| | Color | measured before | Strength | measured before | Modulus |
| | retention | leaving under the | retention | leaving under the | retention |
| | (%) | conditions (g/d) | (%) | conditions (g/d) | (%) |
| Embodiment 11 | −13.0 | 24.3 | 73.5 | 778 | 96.3 |
| Embodiment 12 | −17.5 | 23.5 | 70.2 | 766 | 91.1 |
| Embodiment 13 | −16.3 | 23.9 | 72.3 | 785 | 92.5 |
| Embodiment 14 | −14.3 | 23.7 | 71.7 | 781 | 90.5 |
| Embodiment 15 | −14.0 | 23.9 | 72.4 | 793 | 93.2 |
| Embodiment 16 | −13.9 | 24.3 | 71.9 | 753 | 95.5 |
| Embodiment 17 | −14.1 | 24.1 | 70.8 | 788 | 97.1 |
| Embodiment 18 | −14.3 | 24.0 | 73.1 | 762 | 93.2 |
| Embodiment 19 | −14.9 | 23.6 | 72.9 | 770 | 94.6 |
| Embodiment 20 | −17.8 | 23.7 | 70.1 | 761 | 90.7 |
| Comparative | −19.0 | 24.2 | 72.1 | 762 | 89.7 |
| example 2 | | | | | |

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

1. Aromatic diamine containing 15 ppm or less of impurities.

2. The aromatic diamine according to claim 1, wherein the impurities contain 10 ppm or less of oxidized aromatic diamine.

3. The aromatic diamine according to claim 1, wherein the impurities contain 5 ppm or less of chloroaniline

4. Aramid fiber comprising:
aromatic polyamide polymer by polymerizing aromatic diamine according to claim 1 with aromatic diacid halide; and
stabilizer added to the aromatic polyamide polymer,
wherein a color retention (ΔL) is −18.0˜−12.0.

5. The aramid fiber according to claim 4, wherein the stabilizer contains at least one of organic phosphorous-based stabilizer, phenol-based stabilizer, and hindered amine-based stabilizer.

6. The aramid fiber according to claim 4, wherein the stabilizer is contained in a range from 20 to 10,000 ppm.

7. A method for manufacturing aromatic diamine comprising:
adding a refining supplement to aromatic diamine; and
refining the aromatic diamine with the refining supplement.

8. The method according to claim 7, wherein the refining supplement contains an antioxidant.

9. The method according to claim 8, wherein the antioxidant contains hydrazine compound expressed as the following chemical formula 1,
R₁R₂N—NR₃R₄ [Chemical Formula 1]
wherein ‘R1’, ‘R2’, ‘R3’, and ‘R4’ may be aliphatic group, aromatic group, halogen atom, hydroxyl group, nitro group, nitroso grouop, cyano group, amino group, imino group, azo group, carbonyl group, carboxyl group, acyl group, alkoxycarbonyl group, aryloxycarbonyl group, carbamoyl group, alkoxyl group, aryloxy group, haloalkyl group, mercapto group, alkylthio group, arylthio group, sulfo group, sulphinyl group, sulfonyl group, or heterocyclic group, respectively; and two substituents selected from ‘R1’, ‘R2’, ‘R3’, and ‘R4’ may be cyclic compounds combined discretionally.

10. The method according to claim 9, wherein the refining supplement contains an organic solvent.

11. The method according to claim 10, wherein the boiling point of the organic solvent is lower than the boiling point of the aromatic diamine.

12. The method according to claim 10, wherein the organic solvent is added in a range from 1 time to 100,000 times in comparison to the weight of impurities.

13. A method for manufacturing aramid fiber comprising:
preparing aromatic diamine according to claim 7;
preparing aromatic polyamide polymer by polymerizing aromatic diamine with aromatic diacid halide;
preparing spinning dope by dissolving the aromatic polyamide polymer in a solvent; and
preparing aramid filament by spinning the spinning dope.

14. The method according to claim 13, further comprising adding a stabilizer to the aromatic polyamide polymer before spinning the spinning dope.

15. The method according to claim 13, further comprising coating the aramid filament with the stabilizer after preparing the aramid filament.

16. The method according to claim 14, wherein the stabilizer contains at least one of organic phosphorous-based stabilizer, phenol-based stabilizer, and hindered amine-based stabilizer.

17. Aramid fiber comprising:
aromatic polyamide polymer by polymerizing aromatic diamine according to claim 2 with aromatic diacid halide; and
stabilizer added to the aromatic polyamide polymer,
wherein a color retention (ΔL) is −18.0˜−12.0.

18. Aramid fiber comprising:
aromatic polyamide polymer by polymerizing aromatic diamine according to claim 3 with aromatic diacid halide; and
stabilizer added to the aromatic polyamide polymer,
wherein a color retention (ΔL) is −18.0˜−12.0.

19. A method for manufacturing aramid fiber comprising:
preparing aromatic diamine according to claim 8;
preparing aromatic polyamide polymer by polymerizing aromatic diamine with aromatic diacid halide;
preparing spinning dope by dissolving the aromatic polyamide polymer in a solvent; and
preparing aramid filament by spinning the spinning dope.

20. A method for manufacturing aramid fiber comprising:
preparing aromatic diamine according to claim 9;
preparing aromatic polyamide polymer by polymerizing aromatic diamine with aromatic diacid halide;
preparing spinning dope by dissolving the aromatic polyamide polymer in a solvent; and
preparing aramid filament by spinning the spinning dope.
